(19) United States
(12) Patent Application Publication
(10) Pub. No.: US 2012/0289778 A1
(43) Pub. Date: Nov. 15, 2012

(54) ENDOSCOPE CAPABLE OF DISPLAYING A SCALE FOR DETERMINATION OF THE SIZE OF THE TARGET

(76) Inventor: Chih-Chun CHAN, Taoyuan County (TW)

(21) Appl. No.: 13/166,332

(22) Filed: Jun. 22, 2011

(30) Foreign Application Priority Data
May 10, 2011 (TW) 100116351

(51) Int. Cl. A61B 1/04

(52) U.S. Cl. 600/109

(57) ABSTRACT

An endoscope includes an endoscope body carrying a display device and buttons, a display control circuit, a flexible tube member, an image pickup module, and a stop pin, which has a proximal end thereof connected to the composite of the flexible tube member and the image pickup module and a distal end thereof extending out of the composite to a predetermined length. Further, the display control circuit includes a scale display procedure, which is adapted for displaying a scale on the display device and has sufficient accuracy for determining the size, area or angle of the target only when the distal end of the stop pin is stopped against the target. Thus, the stop pin can be used to establish a distance between the image pickup module and the target, enabling the scale to be used for determination of the size, area or angle of the target being displayed on the display device.

### BACKGROUND OF THE INVENTION

1. Field of the Invention

The present invention relates to endoscopes and more particularly, to such an endoscope, which is capable of displaying a scale for determination of the size of the target.

2. Description of the Related Art

Conventional endoscopes, either for industrial application or medical application, commonly use an image pickup unit at the front end of a flexible tube (the assembly of a flexible tube, lens and light source) to perform illumination and image pickup operations. When picking up the image of a target, the light source is used to illuminate the target object, and the lens is used to pick up the image of the target object. The flexible tube is inserted into the inside of the object. After the flexible tube reaches the target to be examined, the light source illuminates the target, and then the lens picks up the image of the target, and the image thus obtained is transmitted to a display screen for display. Thus, the user can watch the image on the display screen to observe the target that is not directly observed by the naked eyes.

Conventional endoscopes commonly use one single lens to pick up images. In consequence, the images thus obtained are not 3D images. Using non-3D images cannot help determine the size of the target. Even if a scale is displayed on the display screen of the endoscope, the user still cannot accurately determine the size of the target by means of the scale due to that the distance between the target and the lens is unknown.

Taiwan Patent M358624 and Publication No. 201029620 disclose a technique of the installation of a contact probe in the front side of the image pickup lens. By means of attaching the contact probe to the target to maintain a predetermined distance between the image pickup lens and the target, the digital image thus obtained is kept in a predetermined proportion, facilitating further digital image measurement and further distance or area calculation of every part in the digital image. However, this method cannot allow the user to roughly estimate the length or area through visual determination, and a posterior calculation is necessary to obtain the target data. Therefore, there is a room for improvement on the aspect of convenience.

Japanese patent JP2008194156 discloses an endoscope using a probe. According to this design, a retractable probe is provided for stopping against the target when picking up the image of the target. By means of corresponding the distance between the mark on the probe (elements 36 and 36M in FIG. 5) and the end of the probe to the scale on the display device, the correctness of the current scale is determined. According to this prior art design, the probe is retractable and the probe extending distance is not usable as a reference for measurement determination, thus, the measurement determination is complicated. Further, only an experienced person can use the scale, causing inconvenience.

### SUMMARY OF THE INVENTION

The present invention has been accomplished under the circumstances in view. It is the main object of the present invention to provide an endoscope capable of displaying a scale for determination of the size of the target, which uses a stop pin to stop against the target, thereby fixing the distance between the image pickup module and the target for enabling the user to determine the size, area or angle of the target being displayed on the display device.

To achieve this and other objects of the present invention, an endoscope comprises an endoscope body, which comprises a display device and a set of buttons, a display control circuit electrically coupled with the display device and the buttons and adapted for receiving an image signal and displaying the received image signal on the display device and controllable by the user by means of the buttons, a flexible tube connected with one end thereof to the endoscope body, an image pickup module installed in the opposite end of the flexible tube and electrically connected to the display control circuit by an electric wire in the flexible tube member and adapted for picking up the image of a target and transmitting the image signal thus obtained to the display control circuit, and a stop pin, which has a proximal end thereof connected to the composite of the flexible tube member and the image pickup module and a distal end thereof extending out of the composite of the flexible tube member and the image pickup module to a predetermined length. Further, the display control circuit comprises a scale display procedure for displaying a scale on the display device. The scale has sufficient accuracy for determining the size, area or angle of the target only when the distal end of the stop pin is stopped against the target. Thus, the stop pin can be used to establish a distance between the image pickup module and the target, enabling the scale to be used for determination of the size, area or angle of the target being displayed on the display device.

### BRIEF DESCRIPTION OF THE DRAWINGS

FIG. 1 is a schematic architectural view of an endoscope in accordance with a first embodiment of the present invention.

FIG. 2 is a circuit block diagram of the endoscope in accordance with the first embodiment of the present invention.

FIG. 3 is a schematic drawing illustrating an operation status of the endoscope in accordance with the first embodiment of the present invention.

FIG. 4 is a schematic architectural view of a part of an endoscope in accordance with a second embodiment of the present invention, illustrating the shape of the stop pin.

FIG. 5 is another schematic architectural view of a part of the endoscope in accordance with the second embodiment of the present invention, illustrating the shape of an alternate form of the stop pin.

### DETAILED DESCRIPTION OF THE INVENTION

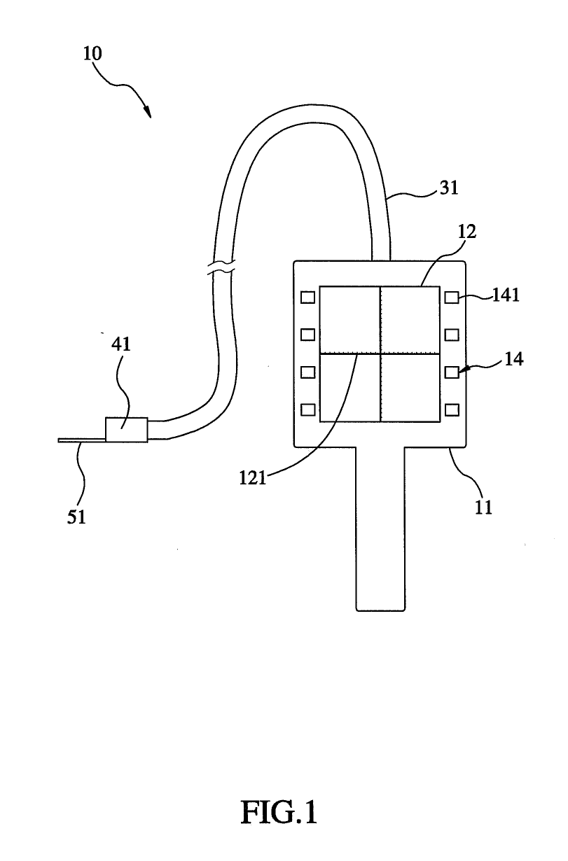

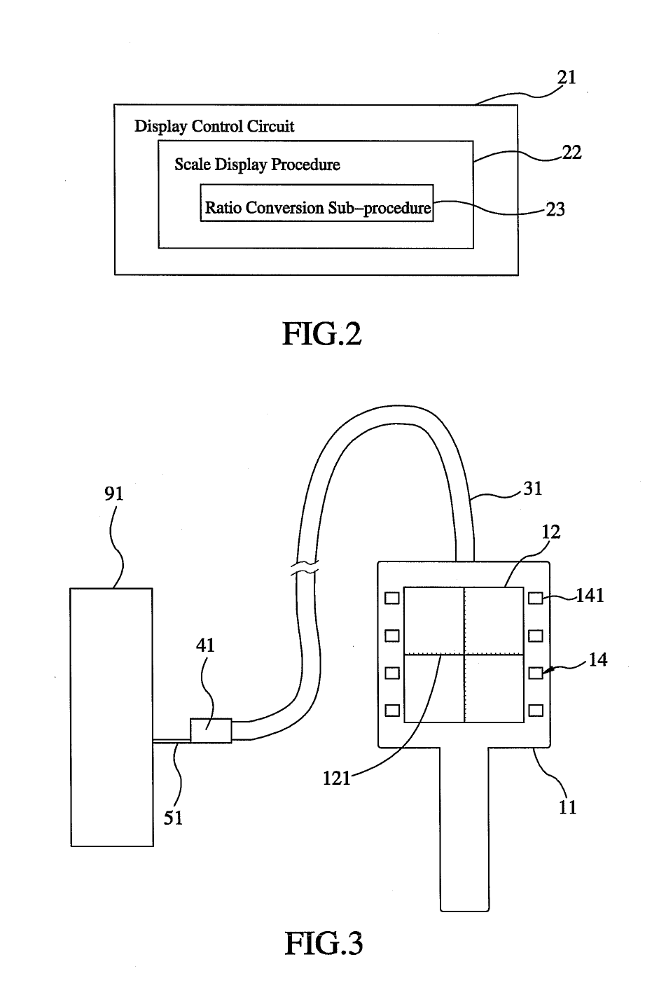

Referring to FIGS. 1 and 2, an endoscope 10 in accordance with a first embodiment of the present invention is shown comprising an endoscope body 11, a display control circuit 21, a flexible tube member 31, an image pickup module 41 and a stop pin 51.

The endoscope body 11 comprises a display device 12 and a set of buttons 14.

The display control circuit 21 is electrically coupled with the display device 12 and the buttons 14 and adapted for receiving an image signal and displaying the received image signal on the display device 12, and controllable by the user by means of the buttons 14. According to this embodiment, the display control circuit 21 is built in the endoscope body 11. However, this installation arrangement is not a limitation. The display control circuit 21 can also be installed in an external device and electrically coupled with the display device 12 and the buttons 14.

The flexible tube member 31 has its one end connected to the endoscope body 11.

The image pickup module 41 is installed in the other end of the flexible tube member 31, and electrically connected to the display control circuit 21 by an electric wire in the flexible tube member 31 (the electric wire conducting technique is of the known art and not shown in the drawing), and adapted for picking up the image of a target 91 and transmitting the image signal thus obtained to the display control circuit 21.

The main features of this embodiment are outlined hereinafter.

The stop pin 51 has its one end connected to the composite of the flexible tube member 31 and the image pickup module 41, and its other end extending out of the composite of the flexible tube member 31 and the image pickup module 41 to a predetermined length.

The display control circuit 21 comprises a scale display procedure 22 for displaying a scale 121 on the display device 12. The scale 121 is accurate for determining the size of the target 91 only when the end of the stop pin 51 is stopped against the target 91. According to this embodiment, the scale display procedure 22 is a software module installed in the display control circuit 21, and controllable by the user or subject to a predetermined procedure instruction to display or not to display the scale 121.

The operation of this first embodiment is outlined hereinafter.

As shown in FIG. 3, when operating the endoscope, employ the scale display procedure 22 to display a scale 121 on the display device 12, and then insert the flexible tube member 31 into position to hold the image pickup module 41 in the image pickup position, and then operate the flexible tube 31 to stop the stop pin 51 against the target 91. Thus, the distance between the image pickup module 41 and the target 91 is established. At this time, the user can use the scale 121 on the display device 12 to determine the size, area or angle of the target 91 being displayed on the display device 12. When determining the area of the target 91, use the known size unit or AOI technique (automated optical inspection) to roughly estimate the area. When determining the angle of the target 91, employ visual inspection or AOI technique to make estimation or software measurement, thereby obtaining the angle data of the target 91. As AOI technique is the known technique that has been widely used for production detection today, no further detailed description in this regard is necessary.

It can be seen that the user can employ the technique disclosed in the first embodiment of the present invention to use the stop pin 51 for establishing the distance between the image pickup module 41 and the target 91 and then to use the scale 121 for determining the size, area or angle of the target 91 being displayed on the display device 12.

Further, in this first embodiment, the end of the stop pin 51 can be within the image pickup range of the image pickup module 41 so that the user can see the image on the display device 12 to visually check whether or not the stop pin 51 has reached the correct or predetermined position.

Further, in this first embodiment, the major axis of the stop pin 51 is kept in a parallel relationship relative to the image pickup direction of the image pickup module 41, facilitating determination of the relationship (vertical, horizontal or oblique relationship) between the surface of the target 91 and the image pickup module 41 by the user.

Further, one of the buttons 14 in this first embodiment can be defined as a scale button 141. The user can operate the scale button 131 to control the display of the scale 121 on the display device 12. Thus, the scale 121 can be displayed on the display device 12 after the stop pin 51 has been stopped against the target 91. Alternatively, the scale 121 can be displayed on the display device 12 before stopping the stop pin 51 against the target 91.

Further, the scale display procedure 22 in accordance with the first embodiment of the present invention further comprises a ratio conversion sub-procedure 23 that defines the specification of the scale 121 when the display device 12 is used at a different display ratio so that the display control circuit 21 can display the matching scale 121 subject to the display ratio of the display device 12. As a regular endoscope has a Zoom in/Zoom out function, the ratio conversion sub-procedure 23 enables the display control circuit 21 to display the matching scale 121 subject to the display ratio of the display device 12 when the user uses the Zoom in/Zoom out function. Thus, the user can accurately determine the size of the target 91 when changes the display ratio.

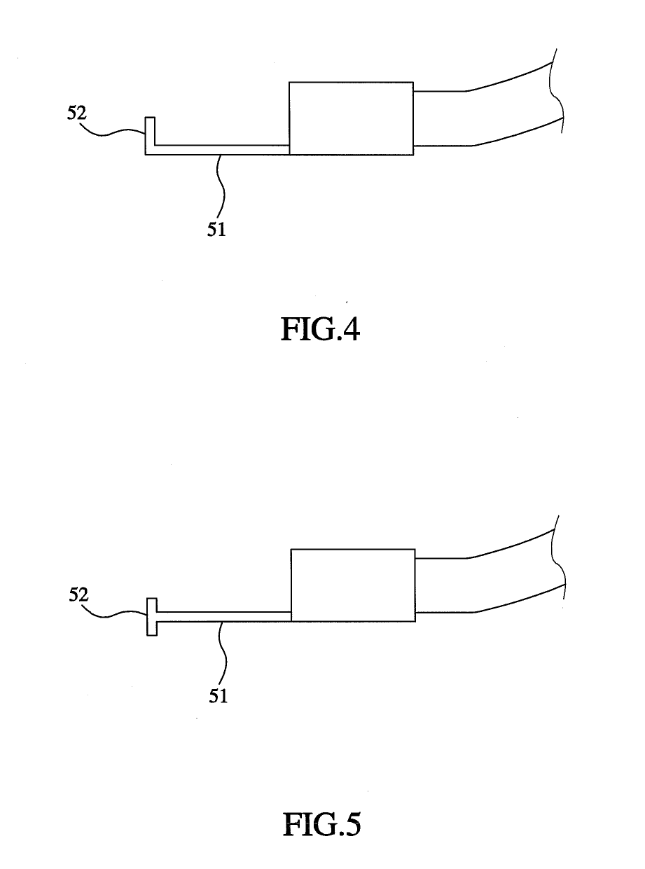

FIGS. 4 and 5 illustrate an endoscope in accordance with a second embodiment of the present invention. This second embodiment is substantially similar to the aforesaid first embodiment with the exception of the following features.

The stop pin 51 has a transverse portion 52 at the distal end thereof. The transverse portion 52 has a predetermined length and defines with the body of the stop pin 51 a predetermined contained angle. According to this second embodiment, the transverse portion 52 extends from the distal end of the stop pin 51 at right angles and is disposed within the image pickup range of the image pickup module 41.

By means of stopping the transverse portion 52 against the target 91, the user can determine the flatness of the surface of the target 91 and the status of perpendicularity of the target 91 relative to the image pickup direction.

Further, assume the length of the transverse portion 52 is known (for example, 0.5 cm), thus, the user can correspond the scale 121 to the image of the transverse portion 52 pm the display device 12 to correct the accuracy of the scale 121.

Further, the stop pin 51, i.e., the combination of the transverse portion 52 and the body of the stop pin 51 can be configured in an L-shaped profile, as shown in FIG. 4, or T-shaped profile, as shown in FIG. 5.

The other structural details and achievable effects of this second embodiment are same as the aforesaid first embodiment, and shall not be described further.

Although particular embodiments of the invention have been described in detail for purposes of illustration, various modifications and enhancements may be made without departing from the spirit and scope of the invention. Accordingly, the invention is not to be limited except as by the appended claims.

What is claimed is:

1. An endoscope capable of displaying a scale for determination of the image size of a target, comprising:
an endoscope body comprising a display device and a set of buttons;
a display control circuit electrically coupled with said display device and said buttons and adapted for receiving an image signal and displaying the received image signal on said display device and being controllable by the user by means of said buttons;
a flexible tube member having one end thereof connected to said endoscope body;
an image pickup module installed in the other end of said flexible tube member and electrically connected to said display control circuit by an electric wire in said flexible tube member and adapted for picking up the image of a target and transmitting the image signal thus obtained to said display control circuit;
wherein said flexible tube member is provided with a stop pin, said stop pin having a proximal end thereof connected to the composite of said flexible tube member and said image pickup module and a distal end thereof extending out of the composite of said flexible tube member and said image pickup module to a predetermined length;
said display control circuit comprises a scale display procedure for displaying a scale on said display device, said scale having sufficient accuracy for determining the size, area or angle of said target only when the distal end of said stop pin is stopped against said target.

2. The endoscope as claimed in claim 1, wherein the distal end of said stop pin is within the image pickup range of said image pickup module.

3. The endoscope as claimed in claim 1, wherein said stop pin has an imaginary long axis thereof kept in a parallel relationship relative to the image pickup direction of said image pickup module.

4. The endoscope as claimed in claim 1, wherein one said buttons is defined as a scale button operable by the user to control the display of said scale on said display device.

5. The endoscope as claimed in claim 1, wherein said scale display procedure further comprises a ratio conversion sub-procedure that defines the specification of said scale for enabling said display control circuit to display said scale subject to the display ratio of said display device.

6. The endoscope as claimed in claim 1, wherein said stop pin comprises a transverse portion disposed at the distal end thereof, said transverse portion having a predetermined length and defining with the body of said stop pin a predetermined contained angle.

7. The endoscope as claimed in claim 6, wherein said transverse portion extends from the distal end of said stop pin at right angles.

8. The endoscope as claimed in claim 6, wherein said transverse portion is within the image pickup range of said image pickup module.
